(19) United States
(12) Patent Application Publication
(10) Pub. No.: US 2005/0249686 A1
(43) Pub. Date: Nov. 10, 2005

(54) Cosmetic composition free of water comprising fatty acid esters, petrolatum oil and non-ionic polymers

(76) Inventors: Francoise Pataut, Paris (FR); Charles Gringore, Paris (FR)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/115,128

(22) Filed: Apr. 27, 2005

Related U.S. Application Data
(60) Provisional application No. 60/620,338, filed on Oct. 21, 2004.

(30) Foreign Application Priority Data
Apr. 27, 2004 (FR) 04 50805

(51) Int. Cl. A61K 7/06

(52) U.S. Cl. 424/70.1

(57) ABSTRACT

This invention relates to a cosmetic non-aqueous composition comprising a non-ionic fixing polymer, petrolatum oil, an alkanol and a fatty acid ester used as a co-solvent, to non-permanently reshape the hair and to a production method of the same.

This application claims benefit of U.S. Provisional Application No. 60/620,338 filed Oct. 21, 2004, the contents of which are incorporated herein by reference. This application also claims benefit of priority under 35 U.S.C. § 119 to French Patent Application No. 04 50805, filed Apr. 27, 2004, the contents of which are also incorporated by reference.

The present disclosure relates to a cosmetic non-aqueous composition comprising at least one non-ionic fixing polymer, petrolatum oil, at least one alkanol, and at least one cosolvent. The composition is used, for example, for non-permanent reshaping of keratin material, such as hair.

The present disclosure relates more particularly to such a cosmetic composition for hair reshaping that also adds shine to it without leaving a ‘synthetic’ feel. Reshaping the hair while providing shine to it has become nowadays a pressing need for consumers. Typically, such reshaping compositions are based on anionic or non-ionic fixing polymers combined with silicones, such as phenyl group containing silicones.

This combination may nevertheless leave a very ‘synthetic’ feel that is very unpleasant, especially for some hair types.

In this regard, patent application JP 54 151140 discloses a composition comprising petrolatum oil, silicone oil, fatty acid esters, in a water-alcohol solution comprising from 5% to 20% water.

It is known that petrolatum oil combined with a non-ionic polymer may provide shine and styled hold to the hair, but an application of an excessive concentration may result in a greasy feeling.

One practical and efficient way to deposit petrolatum oil onto the hair without such drawbacks is by carrying it through an aerosol or non-aerosol spray, resulting in excellent spreading as well as good hair shape retention. However, the vehicle or carrier must quickly evaporate so as to leave an oil deposit on the hair. Such aerosol or non-aerosol sprays must also be capable to deliver a very weakly sternutatory spraying.

Lower C₁-C₆ alkanols, such as ethanol, would be suitable for use as potential vehicles for such petrolatum oil containing compositions, except that the latter is not soluble in these alkanols, and is especially ethanol insoluble.

It would this be desirable to be able to prepare cosmetic compositions that fix the hair while providing shine to it, without leaving a synthetic feel, with excellent spreading and shape retention, wherein these compositions come in the form of aerosol or non-aerosol sprays.

The present disclosure thus relates to such compositions comprising a relatively substantial amount of petrolatum oil, a small amount of silicones, or even no silicone at all, and being free of water as well.

The present disclosure also discloses the preparation of such compositions as aerosol or non-aerosol sprays that can provide the hair with shine and styled hold while counteracting at least one of the drawbacks of prior art.

The present inventors have surprisingly discovered that it is possible to counteract at least one of the drawbacks of prior art by preparing a non-aqueous cosmetic composition comprising at least one non-ionic fixing polymer, petrolatum oil, at least one C₁-C₆ alkanol and at least one fatty acid ester.

A useful amount of oil is thus applied onto the hair once the alkanol-based solvent has been evaporated.

As previously mentioned, the composition according to the present disclosure comprises at least one non-ionic fixing polymer. As used herein, ‘fixing polymer’ is intended to mean any polymer that may convey a shape to the hair or modify the hair shape.

Non-ionic fixing polymers suitable for use in the present disclosure include, but are not limited to:
vinylpyrrolidone homopolymers;
vinylpyrrolidone and vinylacetate copolymers;
polyalkyloxazolines such as polyethyloxazolines commercially marketed by DOW CHEMICAL under the trade names PEOX® 50 000, PEOX® 200 000 and PEOX® 500 000;
vinylacetate homopolymers such as the product marketed under the trade name APPRETAN® EM by HOECHST or the product marketed under the trade name RHODOPAS® A 012 by RHONE POULENC;
vinylacetate and acrylic ester copolymers such as the product marketed under the trade name RHODOPAS® AD 310 by RHONE POULENC;
vinylacetate and ethylene copolymers such as the product marketed under the trade name APPRETAN® TV by HOECHST;
vinylacetate and maleic ester copolymers, such as vinylacetate and dibutylmaleate copolymers, e.g. the product marketed under the trade name APPRETAN® MB EXTRA by HOECHST;
polyethylene and maleic anhydride copolymers;
non-ionic polyurethanes;
alkyl acrylate homopolymers and alkyl methacrylate homopolymers such as the product commercially marketed under the trade name MICROPEARL® RQ 750 by MATSUMOTO or the product commercially marketed under the trade name LUHYDRAN® A 848 S by BASF;
acrylic ester copolymers such as, for example, alkyl acrylate and alkyl methacrylate copolymers such as the products commercially marketed by ROHM & HAAS under the trade names PRIMAL® AC-261 K and EUDRAGIT® NE 30 D, by BASF under the trade names ACRONAL® 601, LUHYDRAN® LR 8833 or 8845, by HOECHST under the trade names APPRETAN® N 9213 or N921 2;
copolymers of acrylonitrile and a non-ionic monomer selected, for example, from butadiene and alkyl(meth)acrylates; included are products marketed under the trade names NIPOL® LX 531 8 by NIPPON ZEON or those marketed under the trade name CJ 0601 8 by ROHM & HAAS;
alkyl acetate and urethane copolymers such as 8538-33 product commercially marketed by NATIONAL STARCH;
polyamides such as ESTAPOR® LO 11 commercially marketed by RHONE POULENC;
non-ionic guar gums, chemically modified or not.

Non-ionic non-modified guar gums include for example products sold under the trade name VIDOGUM® GH 175 by UNIPECTINE and under the trade name JAGUAR® C by MEYHALL.

Non-ionic modified guar gums, suitable for use herein, are, for instance, modified with C₁-C₆ hydroxyalkyl moieties. Suitable examples thereof include hydroxymethyl, hydroxyethyl, hydroxypropyl and hydroxybutyl moieties.

These guar gums are well known in the art and may be prepared, for example, by reacting corresponding alkene oxides, such as, for example, propylene oxides, with guar gum, so as to obtain a hydroxypropyl moiety modified guar gum.

Such non-ionic guar gums optionally modified by hydroxyalkyl moieties include, for example, those commercially marketed under the trade names JAGUAR® HP8, JAGUAR® HP60 and JAGUAR® HP120, JAGUAR® DC 293 and JAGUAR® HP 105 by MEYHALL, or under the trade name GALACTASOL® 4H4FD2 by AQUALON.

Non-ionic fixing polymers suitable for use in the present disclosure may be chosen from non-ionic vinylpyrrolidone-based polymers.

According to one embodiment of the present disclosure, non-ionic fixing polymers are chosen from vinylpyrrolidone homopolymers or vinylpyrrolidone and vinylacetate copolymers.

The at least one non ionic fixing polymer may be present in the disclosed composition in an amount ranging from 0.01 to 8%, such as from 0.1 to 3%, by weight, relative to the total weight of the composition.

The petrolatum oil may be present in an amount ranging from 3 to 20%, such as from 5 to 10% by weight, relative to the total weight of the composition.

The composition according to the present disclosure further comprises at least one alkanol.

Suitable alkanols for use in the composition according to the present disclosure include, but are not limited to, lower C₁-C₆ alkanols such as ethanol, propanol, butanol or mixtures thereof. In one embodiment, the at least one alkanol is ethanol.

The at least one alkanol)may be present in the presently disclosed composition in an amount ranging from 5 to 75%, such as from 10 to 60%, for example from 15 to 50%, by weight, relative to the total weight of the composition.

The composition according to the present disclosure further comprises at least one fatty acid ester.

As used in the context of the present disclosure, ‘fatty acid ester’ is intended to mean a carboxylic acid ester, wherein the acid comprises at least 8 carbon atoms. The acid formula is, for example, RCOOH, wherein R is a saturated or branched hydrocarbon chain containing at least 7 carbon atoms.

In one embodiment, the at least one fatty acid ester according to the present disclosure is chosen from esters of saturated or unsaturated, linear or branched C₈-C₂₈ aliphatic monocarboxylic acids with saturated or unsaturated, linear or branched C₁-C₂₈ monovalent alcohols.

Fatty acid esters suitable for use in the present disclosure include, for example, C₁-C₂₂ alkyl monocarboxylates such as methyl, propyl, butyl, isopropyl or hexyl monocarboxylates.

Amongst C₁-C₂₂ alkyl monocarboxylates there may also be mentioned dihydroabiethyl behenate, octyldodecyl behenate, isocetyl behenate; (iso)stearyl octanoate, isocetyl octanoate, octyl octanoate, cetyl octanoate; decyl oleate; isocetyl isostearate; isocetyl laurate; isocetyl stearate; isodecyl octanoate; isodecyl oleate; isononyl isononanoate, octyl isononanoate, 2-ethylhexyl isononanoate; myristyl stearate; octyl pelargonate; octyl stearate; octyldodecyl erucate, oleyl erucate; octyl palmitate, isostearyl palmitate, ethyl and isopropyl palmitates, ethyl-2-hexyl palmitate, 2-octyldecyl palmitate, C₁-C₁₀ alkyl myristates, preferably C₁-C₆, such as isopropyl myristate, butyl myristate, cetyl myristate; hexyl stearate, butyl stearate, isobutyl stearate; hexyl laurate, 2-hexyldecyl laurate, or mixtures thereof.

For example, in some embodiments, the at least one fatty acid ester is chosen from ethyl and isopropyl palmitates, ethyl-2-hexyl palmitate, 2-octyidecyl palmitate, C₁-C₁₀ alkyl myristates, preferably C₁-C₆ such as isopropyl-, butyl-, and cetyl myristate, 2-octyldodecyl myristate, hexyl stearate, butyl stearate, isobutyl stearate; hexyl laurate, 2-hexyldecyl laurate and isononyl isononanoate, and cetyl octanoate.

A fatty acid ester that is liquid at ambient temperature (25° C.) and at ambient atmospheric pressure is used in at least one embodiment.

The at least one fatty acid ester may be present in the presently disclosed composition in an amount ranging from 1 to 50%, such as from 5 to 35%, for example, from 10 to 30%, by weight, relative to the total weight of the composition.

In order to improve the cosmetic properties of hair fibers or to reduce or prevent their damage, the composition used according to the present disclosure may further comprise at least one cosmetic additive.

The at least one additive is, for example, chosen from hair fiber protecting agents, vitamins, provitamins, fragrances, preservatives, sequestering agents, acidifying agents, alkalinizing agents, as well as mixtures thereof.

The at least one cosmetic additive may be present in the composition in an amount ranging from 0.001 to 20%, such as from 0.1 to 5%, by weight, relative to the total weight of the composition.

In some embodiments of the present disclosure, the compositions do not comprise any silicone, and in other embodiments, the compositions may comprise silicone in small amounts.

These silicones may be volatile or non-volatile, linear or cyclic.

Silicones suitable for use according to the present disclosure include, for instance, polyorganosiloxanes and may come in the form of oils, waxes, resins or gums.

Organopolysiloxanes as useful herein are defined in more detail in Walter NOLL “Chemistry and Technology of Silicones” (1968) Academic Press. They can be volatile or non-volatile in nature.

In at least one embodiment, non volatile silicones, such as polyalkylsiloxanes, polyarylsiloxanes, polyalkylarylsiloxanes, silicone gums and resins, polyorganosiloxanes modified by organofunctional moieties, and mixtures thereof, may be used.

Silicone gums suitable for use according to the present disclosure are, for example, polydiorganosiloxanes having high number average molecular weights ranging from 200 000 to 1 000 000, used alone or in admixture in a solvent. This solvent may be chosen from volatile silicones, polydimethylsiloxane oils (PDMS), polyphenylmethylsiloxane oils (PPMS), isoparaffins, polyisobutylenes, methylene chloride, pentane, dodecane, tridecane or mixtures thereof.

For example, the following organopolysiloxanes may be used:
polydimethylsiloxane,
polydimethylsiloxane/methylvinylsiloxane gums,
polydimethylsiloxane/diphenylsiloxane,
polydimethylsiloxane/phenylmethylsiloxane,
polydimethylsiloxane/diphenylsiloxane/methylvinylsiloxane.

Organopolysiloxane resins suitable for use according to the present disclosure include crosslinked siloxane systems containing the following units:

R₂SiO_{2/2}, R₃SiO_{1/2}, RSiO_{3/2} and SiO_{4/2}, wherein R represents a hydrocarbon moiety containing from 1 to 16 carbon atoms or a phenyl moiety. In one embodiment, R is a lower C₁-C₄ alkyl radical such as a methyl, or a phenyl radical.

Organomodified silicones suitable for use according to the present disclosure include silicones such as defined above, the structures of which comprise at least one organofunctional moiety linked through a hydrocarbon radical.

Suitable examples of organomodified silicones include, for example, polyorganosiloxanes comprising:
polyethyleneoxy and/or polypropyleneoxy moieties having optionally C₆-C₂₄ alkyl moieties such as products called dimethicone copolyol and commercially marketed by DOW CORNING under the trade name DC 1248 or SILWET L 722, L 7500, L 77, L 711 oils ex UNION CARBIDE and alkyl (C₁₂) methicone copolyol commercially marketed by DOW CORNING under the trade name Q2 5200;
amine moieties, substituted or not, such as the products commercially marketed under the trade names GP 4 Silicone Fluid and GP 7100 by GENESEE or products commercially marketed under the trade names Q2 8220 and DOW CORNING 929 or 939 by DOW CORNING. Amine substituted moieties include, for instance, C₁-C₄ aminoalkyl moieties;
thiol moieties such as the products commercially marketed under the trade names “GP 72 A” and “GP 71” by GENESEE;
alkoxylated moieties such as the product commercially marketed under the trade name “SILICONE COPOLYMER F-755” by SWS SILICONES and ABIL WAX 2428, 2434 and 2440 by GOLDSCHMIDT;
hydroxylated moieties such as polyorganosiloxanes with hydroxyalkyl functionality described in French patent application FR-A-85 16334 of formula (I): 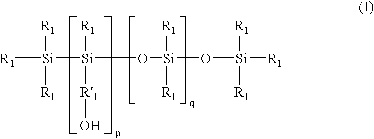
   wherein the R₁ radicals, which may be the same or different, are chosen from methyl and phenyl radicals; at least 60 mole percent of R₁ radicals corresponding to a methyl; R′₁ radical is a divalent hydrocarbon C₂-C₁₈ alkylene chain member; p ranges from 1 to 30; q ranges from 1 to 150;
acyloxyalkyl moieties such as for example polyorganosiloxanes described in U.S. Pat. No. 4,957,732 of formula (II): 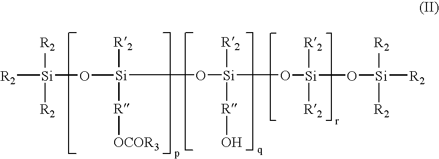
   wherein:

R₂ is a methyl or phenyl, —OCOR₃, or hydroxyl moiety, only one R₂ radical per silicon atom can be OH;

R′₂ is a methyl, a phenyl; at least 60 mole percent of all R₂ and R′₂ radicals being methyl;

R₃ is a C₈-C₂₀ alkyl or alkenyl;

R″ is a linear or branched, divalent hydrocarbon C₂-C₁₈ alkylene radical;

r ranging from 1 to 120;

p ranging from 1 to 30;

q is 0 or less than 0.5 p, p+q being from 1 to 30; polyorganosiloxanes of formula (II) may comprise moieties:

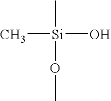

in amounts that do not exceed 15% of the sum p+q+r.
carboxylic-type anionic moieties such as, for example, in products described in patent EP 186 507 to CHISSO CORPORATION, or alkyl carboxylic-type as those present in X-22-3701 E product by SHIN-ETSU; 2-hydroxyalkylsulfonate; 2-hydroxyalkylthiosulfate such as products commercially marketed by GOLDSCHMIDT under the trade names “ABIL S201” and “ABIL S255”.
hydroxyacylamino moieties, such as polyorganosiloxanes described in patent application EP 342 834. Q2-8413 product by DOW CORNING is a suitable example.

In at least one embodiment, polyorganosiloxanes according to the present disclosure are chosen from polyorganosiloxanes comprising polyethyleneoxy and/or polypropyleneoxy moieties having optionally C₆-C₂₄ alkyl moieties such as the product called dimethicone copolyol and commercially marketed by DOW CORNING under the trade name DC 1248 or SILWET L 722, L 7500, L 77, L 711 oils ex UNION CARBIDE and (C₁₂)alkyl methicone copolyol commercially marketed by DOW CORNING under the trade name Q2 5200.

According to the present disclosure, any silicone may also be used in a soluble form, as a dispersion, a microdispersion, emulsion, nanoemulsion or microemulsion.

The composition according to the present disclosure generally comprises less than 1% silicone by weight, relative to the total weight of the composition.

The composition may also comprise at least one cosmetically acceptable additional solvent, chosen, for example, from polyols such as glycerol, propylene glycol, pentanediol, and benzyl alcohol.

The cosmetically acceptable at least one additional solvent according to the present disclosure is present in an amount ranging from 0.001 to 5% by weight of the total composition, such as from 0.1 to 1% by weight, relative to the total weight of the composition.

Hair fiber protecting agents may be any active agent useful to prevent or limit hair damage due to physical or chemical attacks.

Hair fiber protecting agent may thus be chosen from water-soluble, liposoluble or water-insoluble organic UV sunscreens, free-radical scavengers, antioxidants, vitamins, provitamins, as well as mixtures thereof.

Organic UV sunscreens (ultraviolet ray-filtering systems) are chosen, for example, from silicone or non-silicone water-soluble or liposoluble filters and mineral oxide nanoparticles, the surface of which has optionally been treated to become hydrophilic or hydrophobic.

Water-soluble organic UV sunscreens may be chosen from, for example, para-aminobenzoic acid and salts thereof, anthranilic acid and salts thereof, salicylic acid and salts thereof, p-hydroxycinnamic acid and salts thereof, benz-x-azole (benzothioazoles, benzimidazoles, benzoxazoles) sulfonic derivatives and salts thereof, benzophenone sulfonic derivatives and salts thereof, benzylidene camphor sulfonic derivatives and salts thereof, benzylidene camphor derivatives substituted by a quaternary amine and salts thereof, phthalydene-camphor sulfonic acid derivatives and salts thereof, benzotriazole sulfonic derivatives.

Hydrophilic polymers, which have moreover photoprotecting properties against UV radiation due to their chemical nature, may also be used. Included are polymers comprising benzylidene camphor and/or benzotriazole moieties, substituted by sulfonic or quaternary ammonium moieties.

Liposolubles (or lipophilic) organic UV sunscreens suitable for use in the present disclosure include especially p-aminobenzoic acid derivatives, such as esters or amides of p-aminobenzoic acid; salicylic acid derivatives such as esters; benzophenone derivatives; dibenzoylmethane derivatives; diphenylacrylate derivatives; benzofurane derivatives; polymeric UV filters comprising one or more silico-organic residues; cinnamic acid esters; camphor derivatives; trianilino-s-triazine derivatives; urocanic acid ethylic ester; benzotriazoles; hydroxyphenyltriazine derivatives; bis-resorcinol-dialkylaminotriazine; and mixtures thereof.

A liposoluble (or lipophilic) UV filter according to the present disclosure may be chosen from octyl salicylate; 4-tertiobutyl 4′-methoxydibenzoylmethane (PARSOL 1789 ex GIVAUDAN); octocrylene; 2-ethylhexyl 4-methoxy cinnamate (PARSOL MCX) and the compound of following formula (III), or 2-(2H-benzotriazol-2-yl)-4-methyl-6-[2-methyl-3-[1,3,3,3-tetramethyl-1-[(trimethylsilyl)oxy] disiloxanyl]propynyl]phenol, described in patent application EP-A-0 392 883:

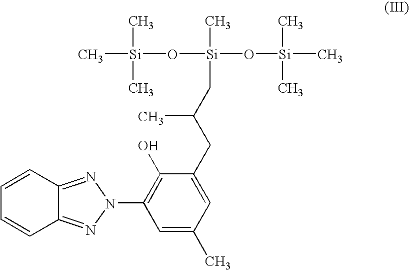

In other embodiments, UV filters according to the present disclosure are benzophenone derivatives such as UVINUL MS 40 (2-hydroxy 4-methoxybenzophenone-5-sulfonic acid) and UVINUL M40 (2-hydroxy-4-methoxybenzophenone) commercially marketed by BASF, benzalmalonate derivatives such as PARSOL SLX (polydimethyl/methyl (3(4-(2,2-bis-ethoxycarbonylvinyl)-phenoxy)-propenyl)siloxane) commercially marketed by GIVAUDAN-ROURE, benzylidene camphor derivatives such as MEXORYL SX ([1-4 divinylbenzene]-b-b′camphor sulfonic acid) prepared by CHIMEX, benzimidazole derivatives such as EUSOLEX 232 (2-phenyl-benzimidazole-5-sulfonic acid) commercially marketed by MERCK.

The pH may be balanced to a suitable value by means of one or more acidifying or alkalinizing agents well known from the state of the art.

Examples of suitable alkalinizing agents include ammoniac, alkaline carbonates, alkanolamines such as mono-, di- and triethanolamines and their derivatives, oxyethylene and/or oxypropylene hydroxyalkylamines and ethylenediamines, sodium or potassium hydroxides and compounds of following formula (IV): 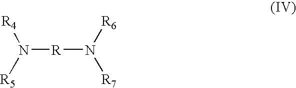
wherein R is a propylene residue optionally substituted by a hydroxyl moiety or a C₁-C₄ alkyl radical; R₄, R₅, R₆ and R₇, which are the same or different, represent a hydrogen atom, a C₁-C₄ alkyl radical or a C₁-C₄ hydroxyalkyl radical.

Acidifying agents are typically, for example, organic or inorganic acids such as hydrochloric acid, orthophosphoric acid, carboxylic acids such as tartaric acid, citric acid, lactic acid, or sulfonic acids.

The composition according to the present disclosure may be pressurized in an aerosol form and may comprise at least one propellant, chosen, for example, from volatile alkanes such as n-butane, propane, isobutane, pentane, carbon dioxide, nitrogen protoxide, dimethylether, nitrogen, compressed air or mixtures thereof.

In at least one embodiment, the propellant is isobutane.

The at least one propellant may be present in the composition in an amount ranging from 25 to 45%, such as from 30 to 40% of the total propellant and composition mixture such as described above.

Also disclosed herein is a method for preparing a cosmetic composition comprising:
a) dissolving petrolatum oil in at least one fatty acid ester; then
b) adding the solution resulting from step a) to a mixture beforehand made of at least one non-ionic fixing polymer and at least one alkanol.

Also disclosed herein is a method comprising pressurizing said cosmetic composition in a can with a propellant.

Also disclosed herein is the preparation of a cosmetic composition that may be used as an aerosol spray as described hereinabove or as a non-aerosol spray, thereby using a dosing pump-dispenser.

In case of an aerosol, the alcohol contained allows for a very slightly sternutatory spraying.

Using the composition according to the present disclosure adds shine to the so treated hair and conveys to the hair a natural feel.

Other than in the operating example, or where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present disclosure. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope of the disclosure are approximations, the numerical values set forth in the specific examples are reported as precisely as possible. Any numerical value, however, inherently contain certain errors necessarily resulting from the standard deviation found in their respective testing measurements.

The present invention will be illustrated in a non-limiting manner with reference to the following example:

### EXAMPLE

A ‘shiny fixative’ aerosol spray was prepared with the following ingredients in grams (for a total of 100 grams):

| | |
|---|---|
| Isopropyl myristate: | 26% |
| Petrolatum oil: | 6.5% |
| OE/OP silicone: | 0.5% |
| Polyvinylpyrrolidone: | 0.65% |
| Ethanol: | 31.35% |
| Isobutane: | 35% |

OE/OP silicone is a silicone comprising oxyethylene and oxypropylene moieties.

After application onto the hair, a good hair shine and styled hold was observed, without traditional greasy and synthetic feel drawbacks.

1. A cosmetic composition, comprising at least one non-ionic fixing polymer, petrolatum oil, at least one lower C₁-C₆ alkanol, and at least one fatty acid ester, wherein said composition is non-aqueous.

2. A cosmetic composition according to claim 1, wherein the at least one non-ionic fixing polymer is chosen from non-ionic vinylpyrrolidone-based polymers.

3. A cosmetic composition according to claim 2, wherein the at least one non-ionic fixing polymer is a vinylpyrrolidone homopolymer.

4. A cosmetic composition according to claim 2, wherein the at least one non-ionic fixing polymer is a copolymer of vinylpyrrolidone and vinylacetate.

5. A cosmetic composition according to claim 1, wherein the at least one non-ionic fixing polymer is present in an amount ranging from 0.01 to 8% by weight, relative to the total weight of the composition.

6. A cosmetic composition according to claim 5, wherein the at least one non-ionic fixing polymer is present in an amount ranging from 0.1 to 3% by weight, relative to the total weight of the composition.

7. A cosmetic composition according to claim 1, wherein the petrolatum oil is present in an amount ranging from 3 to 20% by weight, relative to the total weight of the composition.

8. A cosmetic composition according to claim 7, wherein the petrolatum oil is present in an amount ranging from 5 to 10% by weight, relative to the total weight of the composition.

9. A cosmetic composition according to claim 1, wherein the at least one lower C₁-C₆ alkanol is ethanol.

10. A cosmetic composition according to claim 1, comprising at least one alkanol present in an amount by weight ranging from 5 to 75%, relative to the total weight of the composition.

11. A cosmetic composition according to claim 10, comprising at least one alkanol present in an amount by weight ranging from 15 to 50% by weight, relative to the total weight of the composition.

12. A cosmetic composition according to claim 1, wherein the at least one fatty acid ester is chosen from esters of saturated or unsaturated, linear or branched C₈-C₂₈ aliphatic monocarboxylic acids with saturated or unsaturated, linear or branched C₁-C₂₈ monovalent alcohols.

13. A cosmetic composition according to claim 12, wherein the at least one fatty acid ester is a C₁-C₂₂ alkyl monocarboxylate.

14. A cosmetic composition according to claim 1, wherein the at least one fatty acid ester is liquid at ambient temperature and at atmospheric pressure.

15. A cosmetic composition according to claim 1, wherein the at least one fatty acid ester is chosen from C₁-C₁₀ alkyl myristates.

16. A composition according to claim 15, wherein the at least one fatty acid ester is isopropyl myristate.

17 A cosmetic composition according to claim 12, wherein the at least one fatty acid ester is present in an amount by weight ranging from 1 to 50%, relative to the total weight of the composition.

18. A cosmetic composition according to claim 17, wherein the at least one fatty acid ester is present in an amount by weight ranging from 10 to 30% by weight, relative to the total weight of the composition.

19. A composition according to claim 1, further comprising at least one cosmetic additive chosen from hair fiber protecting agents, vitamins or provitamins, fragrances, preservatives, sequestering agents, acidifying agents, alkalinizing agents, and mixtures thereof.

20. A composition according to claim 19, wherein the at least one cosmetic additive is present in an amount by weight ranging from 0.001% to 20%, relative to the total weight of the composition.

21. A composition according to claim 20, wherein the at least one cosmetic additive is present in an amount by weight ranging from 0.1 to 5% by weight, relative to the total weight of the composition.

22. A composition according to claim 1, further comprising at least one volatile or non-volatile, linear or cyclic silicone.

23. A composition according to claim 22, wherein the at least one silicone is chosen from polyorganosiloxanes.

24. A composition according to claim 23, wherein the polyorganosiloxanes are chosen from polyalkylsiloxanes, polyarylsiloxanes, polyalkylarylsiloxanes, polyorganosiloxanes modified by organofunctional moieties, and mixtures thereof.

25. A composition according to claim 23, wherein the polyorganosiloxane is chosen from polyorganosiloxanes having polyethyleneoxy and/or polypropyleneoxy moieties.

26. A composition according to claim 25, wherein the polyethyleneoxy and/or polypropyleneoxy moieties comprise C₆-C₂₄ alkyl moieties.

27. A composition according to claim 22, wherein the at least one silicone is also used in a soluble form, as a dispersion, a microdispersion, emulsions, nanoemulsions or microemulsions.

28. A composition according to claim 1, comprising less than 1% silicone by weight, relative to the total weight of the composition.

29. A composition according to claim 19, wherein said composition comprises at least one hair fiber protecting agent chosen from water-soluble, liposoluble or water-insoluble organic UV sunscreens, free-radical scavengers, antioxidants, vitamins, provitamins, and mixtures thereof.

30. A composition according to claim 1, further comprising at least one cosmetically acceptable additional solvent chosen from polyols.

31. A composition according to claim 30, wherein said polyols are chosen from glycerol, propylene glycol, pentanediol and benzyl alcohol.

32. A composition according to claim 30, wherein the at least one cosmetically acceptable solvent is present in an amount by weight ranging from 0.001 to 5%, relative to the total weight of the composition.

33. A composition according to claim 32, wherein the at least one cosmetically acceptable solvent is present in an amount by weight ranging from 0.1 to 1%, relative to the total weight of the composition.

34. A composition according to claim 1, wherein said composition is in the form of a spray.

35. A composition according to claim 34, wherein said spray is an aerosol spray comprising at least one propellant.

36. A composition according to claim 35, wherein said at least one propellant is chosen from volatile alkanes, carbon dioxide, nitrogen protoxide, dimethylether, nitrogen, compressed air and mixtures thereof.

37. A composition according to claim 36, wherein said volatile alkanes are chosen from n-butane, propane, isobutane, and pentane.

38. A method of making a non-aqueous cosmetic composition comprising at least one non-ionic fixing polymer, petrolatum oil, at least one lower C₁-C₆ alkanol, and at least one fatty acid ester, said method comprising:
a) combining at least one non-ionic fixing polymer and at least one alkanol to form a mixture;
b) dissolving said petrolatum oil in said fatty acid ester; and
c) adding the solution resulting from step b) to the mixture from a).

39. A method according to claim 38, further comprising pressurizing said cosmetic composition in a can with a propellant.

40. A method for providing shine to hair comprising applying to the hair a non-aqueous composition comprising at least one non-ionic fixing polymer, petrolatum oil, at least one lower C₁-C₆ alkanol, and at least one fatty acid ester.

41. The method according to claim 40, wherein the hair to which said composition has been applied has a natural feel.
